(19) United States
(12) Patent Application Publication
(10) Pub. No.: US 2022/0306813 A1
(43) Pub. Date: Sep. 29, 2022

(54) PULVERULENT SUBSTRATE OPTAINED BY STEAM CRACKING OF A BIOMASS WIITHOUT CHEMICAL AUZILIARY AGENT, AND USES THEREOF

(71) Applicant: EUROPEENNE DE BIOMASSE, Paris (FR)

(72) Inventors: Jean-Baptiste Marin, Paris (FR); Thomas Habas, Paris (FR); Adriana Quintero-Marquez, Le Vesinet (FR); Frédéric Martel, Riems (FR)

(21) Appl. No.: 17/594,949

(22) PCT Filed: Apr. 30, 2020
(86) PCT No.: PCT/FR2020/050729

§ 371 Date: Nov. 3, 2021

(30) Foreign Application Priority Data
May 3, 2019 (FR) FR1904681

(51) Int. Cl. C08H 8/00; C08L 97/02; C12P 19/04

(52) U.S. Cl. CPC C08H 8/00; C08L 97/02; C12P 19/04

(57) ABSTRACT

An energy substrate is obtained by steam cracking of a biomass. More specifically, the disclosure relates to a steam-cracked biomass in the form of dry powder and devoid of any chemical additive, to a method for the production thereof and to the use thereof as a substrate for green chemistry and biotechnological methods such as enzymatic hydrolysis and fermentation.

### CROSS-REFERENCE TO RELATED APPLICATIONS

This application is a national phase entry under 35 U.S.C. § 371 of International Patent Application PCT/FR2020/050729, filed Apr. 30, 2020, designating the United States of America and published as International Patent Publication WO 2020/225504 A2 on Nov. 12, 2020, which claims the benefit under Article 8 of the Patent Cooperation Treaty to French Patent Application Serial No. 1904681, filed May 3, 2019.

### TECHNICAL FIELD

The disclosure relates to an energy substrate obtained by steam-cracking of a biomass. More specifically, the disclosure relates to a steam-cracked biomass in the form of dry powder and devoid of any chemical additive, to a method for the production thereof, and to the use thereof as a substrate for green chemistry and biotechnological methods such as enzymatic hydrolysis and fermentation.

### BACKGROUND

Biotechnological methods or methods of green chemistry use carbon substrates originating from plant foodstuffs raw materials, produced intensively and at high cost. The LCA (life cycle analysis) of the substrate, as well as the food competition and the price of the raw material, are the brakes on the development of these biosourced products, and the bioeconomy in general. The use of “second generation” (2G) lignocellulosic biomass (wood, agricultural waste, co-products of agriculture and of the agro-industry) would make it possible to reduce the environmental impacts (fossil CO₂ emission, fertilizers, phytosanitaries), and the use competition and price competition in the field of foodstuffs.

A number of projects on this subject have been launched since 2006 (120 globally). The principle is the water vapor explosion of the biomass. Most add a chemical auxiliary such as sulfuric acid or alkaline ammonia. All use a “wet” method—the biomass is wetted at the rate of 40 to 70% water. Once the explosion has been carried out, the material is either detoxified or used directly, adding enzymes that hydrolyze the sugar polymers, then simultaneously or subsequently microorganisms, in order to convert the released sugars into molecules of interest.

The steam-cracking differs from hydrothermal pre-treatment, also referred to as aqueous fractionation, solvolysis, hydrothermolysis, or hydrothermal treatment, in that the latter involves using water at a high temperature and high pressure in order to promote the disintegration and the separation of the lignocellulosic matrix.

Thus, industrial-scale methods for transformation of lignocellulosic biomass, which have a large-scale technical reality and are economically viable, as well as having a risk-free good environmental record, are rare. Ethanol is one of the only biotechnological commodities that attempts to start up, today, on a lignocellulosic basis.

The document WO2013/018034 A1 relates to a method for producing a substrate for growing mushrooms. The production of the substrate can be carried out by using various treatment methods, and in particular that of vapor explosion. During use of the vapor explosion method, the lignocellulosic material is subjected to pulverization and is subsequently placed in a reactor that heats the biomass, by virtue of the vapor, to temperatures of from 160° C. to 230° C. The reactor reaches a pressure of between 12 and 28 atmospheres, and then the atmospheric pressure is immediately reduced to atmospheric pressure, creating a vapor explosion.

The document WO2013/105034 describes a method for treating lignocellulosic biomass in order to obtain a liquid composition. The treatment of the biomass is characterized by a step of soaking in order to obtain a solid fraction and a liquid fraction. Subsequently, a portion of the liquid fraction is separated, while the other fraction (the solid fraction and a portion of the liquid fraction) undergoes a vapor explosion step on the solid fraction in order to again obtain a solid and liquid fraction. Finally, the newly obtained liquid fraction is mixed with the solid fraction obtained in the first step.

The document WO2013/152771 relates to a method for treating a lignocellulosic biomass in order to produce biofuels such as ethanol, butanol, hydrogen, methanol, and biogas. This method uses a technique based on the properties of mechanical vapor explosion, by mechanical diabatic decomposition, and under pressure.

The documents FR 2 997 094 A1 and WO 2014/060673 A1 describe, respectively: a method for production of ethanol, referred to as “second generation,” from lignocellulosic biomass, and a method for production of alcohols, referred to as “second generation,” from lignocellulosic biomass. These methods comprise various steps, i.e.: pre-treatment in a reactor, by vapor explosion, following a step of enzymatic hydrolysis of the pre-treated substrate, then a step of ethylic fermentation of solubilized sugars, then extraction of ethanol or alcohols from the fermented effluent, and recycling, upstream or in the pre-treatment reactor, of an internal aqueous flow comprising ethanol or alcohol.

The documents WO2014/204519 A1 and WO2013/191897 A1 describe the treatment of the biomass using vapor explosion methods, as pre-treatment before gasification or combustion.

Finally, the document EP 3 054 050 A describes a continuous treatment method of a lignocellulosic feedstock in order to produce a dehydrated lignocellulosic feedstock having a humidity content in the first target range, which is from 65% to 85%. It describes a method that may involve a step of vapor explosion at a severity factor of between 2.8 and 5.3.

The majority of the known methods are hampered by technical and economic problems: the use of chemical auxiliaries (acidic or alkaline) at a usage cost; these are used, for example, for pre-treatment by increasing the severity, and for neutralization of the pH with the view to the use of a biological auxiliary (enzymes, microorganisms). Moreover, the auxiliaries accentuate the chemical breakdown of the sugars, resulting in a loss of yield and the creation of inhibitors of the microorganisms and the enzymes used in the biotechnological methods or green chemistry, requiring an over-consumption thereof, or pollutants to be separated from the final product. Moreover, even in the absence of auxiliaries, the dilution of the biomass by impregnation causes an increase in the energy costs of heat treatment, and a lower substrate titer, the dilution of which is costly during purification processes (such as extraction or distillation). Finally, these methods are expensive in terms of capital expenditure (tangible and intangible) devoted to the purchase of professional equipment (CAPEX) and operating expenses (OPEX).

Moreover, in the solutions proposed by the prior art, the severity factor is not an established parameter. However, the severity factor is significant because it makes it possible to obtain different compounds, depending on the intensity thereof. Moreover, the solutions of the prior art propose methods that are not carried out continuously, having high humidity levels for the initial biomass. Until today, there is no biomass preparation method in existence for high added-value application that would be viable from an industrial and economic perspective.

### BRIEF SUMMARY

The present disclosure provides a carbon substrate devoid of added acid or alkaline compounds, which is “ready-to-use” for implementation in biotechnological methods, chemistry methods, or green chemistry methods. This substrate is prepared by virtue of a continuous steam-cracking method of a dry lignocellulosic biomass and without adding any chemical auxiliary.

The objective is also that of reducing the production costs, for example, in parallel with a primary use (black pellets), by removing an intermediate product from the production (powder or “granulettes,” i.e., pellets of average compression density), and of using it as a hydrolysis substrate (sugars for green chemistry) or a substrate for hydrolysis and fermentation (sugars for biotechnology).

The disclosure thus relates to a pulverulent carbon substrate of chemical reaction, obtained by continuous steam-cracking of a lignocellulosic biomass without any chemical auxiliary, a composition comprising a substrate of this kind, as well as the uses thereof.

The method is economically viable for commodities such as energy, and thus a fortiori viable for products of higher added value. The carbon substrate obtained is stable.

The economic aspect is essential—continuous and dry operation makes it possible to reduce the sizes of the pieces of equipment (continuous flow) and the volume thereof to be treated (dry flow), and thus the CAPEX; the absence of technological chemical auxiliaries limits the losses due to deterioration, and costs less in terms of purchase costs and neutralizing charge, and in terms of pollution to be treated (OPEX); this also protects the equipment from corrosion (CAPEX). The incoming biomass has undergone only thermal and mechanical modification and, therefore, has not been adjuvanted. It thus preserves its “natural” primary quality; furthermore, it is less expensive to produce than when using wet chemical methods (40% to 70% humidity) in heat treatment. Finally, it stems from a viable and robust installed industrial method, which allows for access to a biomass prepared by steam-cracking, at volumes that allow for economies of scale, and thus OPEX and CAPEX reductions.

Another advantage of the method for producing the pulverulent substrate according to the disclosure is that it does not generate effluents, because it does not comprise any chemical treatment (in particular acidic). The substrate is stable, which allows for the storage and transport thereof. It can be enzymatically hydrolyzed from 50% to 70%, despite the absence of acidic or alkaline pre-treatment. Finally, the product is inexpensive, does not require use of water or effluent, it is derived from an inexpensive commodity product, and can be used to produce high added-value products.

The pulverulent carbon substrate according to the disclosure is advantageously used for the production of sugars and of co-products such as lignin.

The steam-cracked biomass in powder form can be utilized in sugars such as xylose by chemical release, as well as the pre-treated cellulosic portion that can be hydrolyzed by cellulolytic enzymes to form simple sugars; these sugars can then be transformed by chemistry or by bioconversion/fermentation into higher added-value molecules in the fields of biotechnology and green chemistry.

### DETAILED DESCRIPTION

A first object of the disclosure relates to a pulverulent carbon substrate of chemical or biochemical reaction, obtained by continuous steam-cracking of a lignocellulosic biomass at a humidity level of between 5% and 27% without any chemical auxiliary.

In a preferred embodiment, the steam-cracking is carried out by applying a severity factor of between 3 and 5.

Within the meaning of the present disclosure, “chemical reaction” means any reaction, including biochemical reactions such as fermentation, enzymatic hydrolyses, biotechnological methods, in addition to conventional chemical transformations. The concept of a chemical reaction does not include a combustion reaction.

Within the meaning of the present disclosure, “pulverulent substrate” means a substrate in powder form or in the form of pellets, or pellets having a low degree of compression, also referred to as “granulettes.” The granulettes correspond to a form of compressed powder, so as to give the powder the form of a pellet, but that rapidly releases a powder by soaking (little pelletization). This form can be adopted during packaging of the product in order to facilitate the handling thereof, but the characteristics thereof are those of a powder from the moment the substrate is impregnated by a solution (enzymatic, acidic, etc.).

The biomass powder obtained by steam-cracking comprises at least 50% pulverulent compounds having a cross section of less than 0.5 millimeters, and at least 10% of fibrous compounds having a length of over 1 millimeter.

The pellets are cylindrical in shape. The length thereof may be defined as being 99.9% less than 5 cm, and 99.0% less than 4 cm, and less than 10% less than 1 cm. Furthermore, at least 99.0% of the pellets have a diameter of greater than or equal to the diameter selected, i.e., for example, 6, 8 or 10 mm. Finally, the bulk density thereof (with a cylinder having a volume of 5 liters of pellets, tapped 3 times by falling from a height of 20 cm) is between 600 g/l and 700 g/l.

The granulettes have dimensions equivalent to those of the pellets, but have a density of less than 600 g/l, generally between 300 and 600 g/l.

The pulverulent substrate is particularly suitable for implementing biochemical reactions such as enzymatic hydrolysis, fermentation, or any other chemical or biochemical reaction.

The fact that the substrate is present in powder form implies that it is in dry form, preferably between 5% and 27% humidity. This feature distinguishes it from other forms of biomass used for biochemical reactions, which are in liquid form. Indeed, the biomasses of the prior art are impregnated, in particular with chemical auxiliaries, prior to treatment, and are treated in a liquid environment following steam-cracking, in order to eliminate the auxiliaries. Even if presentation in liquid or wet form may be suitable for biochemical applications, this is problematic for preserving the biomass, which will necessarily ferment.

The substrate according to the disclosure thus has the advantage of being dry, by virtue of a method of preparation by means of steam-cracking without prior impregnation, i.e., from biomass having a humidity level that is preferably between 5 and 27% (directly, or optionally after drying). Moreover, the method does not involve the addition of chemical auxiliaries (or additives), producing a clean substrate.

Within the meaning of the disclosure, a “chemical auxiliary” means any compound or any solution that may rest in the steam-cracked product or in the effluents. The auxiliaries create impurities in the steam-cracked product, in terms of the uses thereof and pollutants in the effluents to be rejected. Chemical auxiliaries of this kind are, for example, acids, bases, organic solvents or organic molecules, salts, etc. Products such as lime, carbon dioxide, and renewable dissociated forms of carbon dioxide are not considered chemical auxiliaries, because they are inert with respect to the intended uses and the environment.

The severity factor depends on the pressure, the temperature, and the treatment duration. In a preferred embodiment, a severity factor is between 3 and 5. In a particular embodiment of the disclosure, the severity factor corresponds to a treatment for several minutes (generally between 5 and 30 minutes).

A second object of the disclosure relates to a “ready-to-use” dry composition comprising a pulverulent substrate as defined above, and at least one enzyme.

The dry composition can be preserved and transported; it is stable.

The enzymes allow for hydrolysis of the biomass, once it is impregnated, by the user. Indeed, the enzymes in the dry environment (the biomass in powder form) are inactive, and the activation thereof is initiated by wetting, indeed soaking, the composition.

The enzymes that can be associated with the biomass in a composition of this kind can, for example, be selected from cellulases, a beta-glucosidase, hemicellulases, etc.

A third object of the disclosure relates to a “ready-to-use” dry composition comprising a pulverulent substrate as defined above, and at least one microorganism.

The dry composition can be preserved and transported; it is stable.

The microorganisms allow for the fermentation of the biomass, once it is impregnated, by the user. Indeed, the microorganisms are not active in the dry environment (the biomass in powder form), and the metabolism thereof is activated by wetting, indeed soaking, the composition.

The microorganisms associated with the substrate may be of different types, in particular a microbial biomass or microalgae. The microbial biomass may contain bacteria, yeasts, fungi, or any other type of cell.

In a particular embodiment, the disclosure relates to a “ready-to-use” dry composition comprising a pulverulent substrate, at least one enzyme, and at least one microorganism.

A third object of the disclosure relates to the use of a substrate as defined above, as a support for a chemical reaction.

In a particular embodiment, the substrate is used as a production support for the culture of microorganisms.

It is a case of using the substrate in order to provide the microorganisms, making up the biomass, with the nutrients necessary for the multiplication thereof. It is sufficient to mix a sample of biomass with the substrate in appropriate conditions (humidity, temperature, etc.) in order to grow microorganisms.

A fourth object of the disclosure relates to a method for obtaining sugars from lignocellulosic biomass, consisting in subjecting a pulverulent carbon substrate to enzymatic hydrolysis, or in cultivating a “ready-to-use” dry composition comprising a pulverulent substrate and enzymes.

The substrate obtained from biomass may undergo enzymatic hydrolysis in order to provide high added-value sugars such as xylose, glucose, etc.

The enzymatic and fermentation reactions furthermore generate co-products that must be able to be exploited, such as the hydrolysis or fermentation residue, which contains lignin and fibers having a significant LCV, resinous or terpenic derivatives, phenolic compounds (coumaric, ferulic), condensates rich in furfuraldehyde (polymerization monomer), in acetic and formic acid, etc. The soluble or insoluble lignin co-products can be used as materials (resins, binding agents, feedstocks).

A fifth object of the disclosure relates to a method for obtaining molecules of interest from lignocellulosic biomass, that includes (i) subjecting a pulverulent carbon substrate to fermentation, or (ii) cultivating a “ready-to-use” dry composition comprising a pulverulent substrate and at least one microorganism, or (iii) subjecting a pulverulent substrate to a (conventional) chemical transformation method.

The molecules of interest that can be obtained are intended, for example, for the field of bioenergies (biofuel oils, biogas, etc., such as bioethanol, biomethanol, biomethane, etc.) or bioplastics (biomaterials, biocomposites) or bioproducts (proteins, solvents, any other chemical molecule, etc.).

The substrate according to the disclosure can be used in numerous applications: by manufacturers seeking to produce bioethanol and biobutanol and isobutene and farnesene to make liquid biofuels (biofuel oils in particular, toward light-duty or heavy-duty vehicles such as BP, SHELL, toward aviation such as TOTAL); by biotechnologists wishing to ferment 2G sugars (cellulosic glucose and xylose) toward basic synthons (building blocks) of industry, bioplastics or bioproducts (methane, methanol, formic acid, formol, ethanol, ethylene, acetic acid, oxalic acid, ethanal, propanol, propanediol, acetone, propionic acid, lactic acid, maleic acid, malic acid, fumaric acid, succinic acid, butanol, butanediol, isobutene, butyric acid, hydroxybutyric acid, valeric acid, glutaric acid, capric acid, caproic acid, caprylic acid, amino acids, etc.); by producers of microorganisms (start-up or spinoff) who plan to produce microbial biomass of non-food origin (yeasts rich in proteins, or in oils, or heterotrophic microalgae without light).

A sixth object relates to a continuous preparation method of a pulverulent substrate of chemical reaction as defined above, by steam-cracking of a lignocellulosic biomass, characterized in that the method is carried out:
at a humidity level of between 5 and 27%, and
without a chemical auxiliary.

In a preferred embodiment, the method allows for the preparation of a pulverulent substrate of chemical reaction, except for combustion.

### EXAMPLES

### Example 1: Implementation of a Steam-Cracking Method Making it Possible to Obtain a Pulverulent Carbon Substrate

The production of a pulverulent carbon substrate according to the present disclosure can be achieved proceeding from wood, by implementing the following steps:
obtaining wood fragments of wood of a size of between 0.5 and 14 mm having a humidity level of between 5 and 27%;
continuously introducing a predetermined volume per minute of the wood fragments obtained, into a pressurized reactor, the reactor being fed with substantially saturated water vapor, the pressure of which is between 10 and 25 bar, and the temperature of which is between 180° C. and 220° C.;
exposing wood fragments, introduced into the reactor, to the water vapor for a period sufficient to achieve steam-cracking of between 5 and 30 minutes, the value of the exposure period and the value of the temperature of the substantially saturated vapor being selected such that the severity factor is between 3 and 5, preferably between 3.5 and 4;
continuously extracting, from the reactor, the same predetermined volume of wood fragments per minute, through a plurality of openings leading into a channel that is substantially at atmospheric pressure, so as to bring about explosive decompression of the wood fragments extracted from the reactor in the channel;
separating the steam-cracked powder and the residual vapor extracted from the reactor, the steam-cracked wood powder obtained following separation forming the pulverulent substrate.

1. A pulverulent carbon substrate of chemical or biochemical reaction, formed by a process comprising continuous steam-cracking of a lignocellulosic biomass at a humidity level of between 5% and 27% without any chemical auxiliary, by applying a severity factor of between 3 and 5.

2. A ready-to-use dry composition, comprising:
a pulverulent substrate as defined in claim 1; and
at least one enzyme.

3. A ready-to-use dry composition, comprising:
a pulverulent substrate as defined in claim 1; and
at least one microorganism.

4. A ready-to-use dry composition, comprising:
a pulverulent substrate as defined in claim 1;
at least one enzyme; and
at least one microorganism.

5. The composition of claim 3, wherein the at least one microorganism is a microbial biomass.

6. The composition of claim 3, wherein the at least one microorganism is micro-algae.

7. A method, comprising:
providing a substrate as defined in claim 1; and
using the substrate as a support for a chemical reaction.

8. The method of claim 7, wherein using the substrate as a support for a chemical reaction further comprises using the substrate as a production support for a culture of microorganisms.

9. A method for obtaining sugars from lignocellulosic biomass, comprising:
(i) subjecting a pulverulent carbon substrate as defined in claim 1 to enzymatic hydrolysis, or
(ii) cultivating a composition comprising a pulverulent carbon substrate as defined in claim 1 and one or more of a microorganism, an enzyme, a microbial biomass, or a micro-algae.

10. A method of producing molecules of interest, comprising:
(i) subjecting a pulverulent carbon substrate as defined in claim 1 to fermentation; or
(ii) cultivating a composition comprising a pulverulent carbon substrate as defined in claim 1 and one or more of a microorganism, an enzyme, a microbial biomass, or a micro-algae.

11. The method of claim 10, wherein the molecules of interest are intended to be used in the field of bioenergies or bioplastics or bioproducts.

12. A method of continuous preparation of a pulverulent substrate for a chemical reaction, other than combustion, comprising: continuously steam-cracking a lignocellulosic biomass at a humidity level of between 5% and 27% without any chemical auxiliary while applying a severity factor of between 3 and 5 to the lignocellulosic biomass.

13. A method of forming a pulverulent substrate of chemical reaction, comprising continuously steam-cracking a lignocellulosic biomass at a humidity level of between 5% and 27% without any chemical auxiliary while applying a severity factor of between 3 and 5 to the lignocellulosic biomass
